(19) United States
(12) Patent Application Publication
(10) Pub. No.: US 2008/0287809 A1
(43) Pub. Date: Nov. 20, 2008

(54) Biological information measuring apparatus and biological information measuring method

(75) Inventors: Kazuhiko Amano, Tokyo (JP); Yuji Kato, Hokkaido (JP); Koichi Shimizu, Hokkaido (JP)

Correspondence Address: OLIFF & BERRIDGE, PLC, P.O. BOX 320850, ALEXANDRIA, VA, 22320-4850, US

(73) Assignees: SEIKO EPSON CORPORATION, Tokyo (JP); NATIONAL UNIVERSITY CORPORATION HOKKAIDO UNIVERSITY, SAPPARO-SHI (JP)

(21) Appl. No.: 12/081,924

(22) Filed: Apr. 23, 2008

Related U.S. Application Data
(60) Provisional application No. 60/913,711, filed on Apr. 24, 2007.

(30) Foreign Application Priority Data
Mar. 19, 2008 (JP) 2008-072431

(51) Int. Cl. A61B 5/00

(52) U.S. Cl. 600/476

(57) ABSTRACT

A biological information measuring apparatus measures information of a living body by irradiating the living body with light. The apparatus includes: a first light source irradiating the living body with light; a second light source irradiating the living body with light; and a light receiving section constituted by a plurality of light receiving elements that receives reflected light reflected by the living body or transmitting light transmitting through the living body, during light irradiation of the living body. In the biological information measuring apparatus, the first light source and the second light source are disposed so that a first distance between the light receiving section and the first light source is different from a second distance between the light receiving section and the second light source.

### CROSS-REFERENCE TO RELATED APPLICATION

This application is entitled to the benefit of Provisional Patent Application No. 60/913,711, filed on Apr. 24, 2007.

### BACKGROUND

1. Technical Field

The present invention relates to a biological information measuring apparatus and a biological information measuring method for measuring a tissue of a living body with depth selectivity by using a plurality of light sources.

2. Related Art

In recent years, biometric authentication using biological information, such as a fingerprint, the iris, or a vein pattern, of a regular user is performed to authenticate the identity of regular user, as disclosed in Japanese Patent Publication No. 3549795.

In addition, various kinds of biological information acquisition apparatuses are used to perform biometric authentication.

Some of the biological information acquisition apparatuses are known to include a light source that irradiates light and a light receiving element that receives light.

In addition, light from the light source is irradiated onto a living body, light from the living body is received, and the received light is converted into an electrical signal, and as a result, biological information can be acquired.

Here, since electronic apparatuses that need authentication, such as personal digital assistants and personal computers, are widespread today, a biological information acquisition apparatus that is of large size and whose power consumption is high is not wanted.

On the other hand, as security awareness increases, even higher accuracy of authentication has been requested. Accordingly, it is also demanded to improve the accuracy of biological information.

However, there is a problem in that the quality or accuracy of biological information acquired decreases since a dynamic range of illuminated light or received light is limited.

In particular, as limitation in a light source or a light receiving element used in a small biological information acquisition apparatus becomes severe, there is a problem in that it is difficult to use a high-performance light source or light receiving element in terms of dynamic range and the like.

In order to solve such a problem, a biological information acquisition apparatus disclosed in Japanese Unexamined Patent Application, First Publication No. 2007-330769 that can acquire highly accurate biological information and can be easily made small is known.

However, a dynamic range of illuminated light is reduced if the biological information acquisition apparatus is made small. As a result, since a usable range is limited, there is a problem in that the quality of acquired biological information degrades.

In order to solve such a problem, a biological information acquisition apparatus disclosed in Japanese Unexamined Patent Application, First Publication No. 2007-313295 that can acquire highly accurate biological information and can be easily made small is known.

Here, an optical body measurement technique in the case of a scattering medium, such as a living body, is very important as new measurement for acquiring living body function information using an X-ray CT (computed tomography) or the like.

For this reason, studies of optical CT (computed tomography), optical topography, and the like are actively conducted.

On the other hand, in a vein image acquisition apparatus that has been drawing widespread attention as a biological information acquisition apparatus, a transmissive or reflective optical imaging technique is used.

However, these methods have the following problems.

Although the depth selectivity is given in the optical CT from the point of view that a tomographic image can be obtained, a light emitting element and a light receiving element capable of performing time division measurement with picosecond order are required and the apparatus is very complicated, large sized, and expensive.

In addition, since there is no depth selectivity in the case of optical topography or a typical vein image acquisition apparatus, there is a problem in that information on a blood vessel positioned at a predetermined depth cannot be acquired.

On the other hand, an optical coherence tomography may be mentioned as a method having the depth selectivity. In this case, however, there is a problem in that a reaching depth in a general tissue of a living body is about 1 mm and the use is significantly limited.

### SUMMARY

An advantage of some aspects of the invention is to provide a biological information measuring apparatus and a biological information measuring method for measuring a tissue of a living body with depth selectivity by using a plurality of light sources.

A first aspect of the invention provides a biological information measuring apparatus for measuring information of a living body by irradiating the living body with light, the apparatus including: a first light source irradiating the living body with light; a second light source irradiating the living body with light; and a light receiving section constituted by a plurality of light receiving elements that receives reflected light reflected by the living body or transmitting light transmitting through the living body, during light irradiation of the living body. In the biological information measuring apparatus, the first light source and the second light source are disposed so that a first distance between the light receiving section and the first light source is different from a second distance between the light receiving section and the second light source.

According to this constitution, since the two light sources irradiating the light onto the living body and the light receiving section receiving the reflected light that is reflected by the living body or transmitting light transmitting through the living body during light irradiation of the living body is included in the biological information measuring apparatus, and since the first distance between the light receiving section and the first light source is different from the second distance between the light receiving section and the second light source, it is possible to measure a tissue of a living body having depth selectivity.

Furthermore, this constitution is configured so that light intensity distribution information of a depth in the living body to a point to be measured is acquired by calculating in a image processing based on first light intensity distribution information that is acquired when the first light source is only lit and second light intensity distribution information that is acquired when the second light source is only lit. It is thereby possible to simplify the constitution of the biological information measuring apparatus. Also, the biological information measuring apparatus can be decreased in size.

It is preferable that, in the biological information measuring apparatus of the first aspect of the invention, the first distance and the second distance be determined depending on a depth in the living body to a point to be measured.

According to this constitution, by adjusting the first distance and the second distance, it is possible to desirably obtain the received light intensity distribution data of at the depth to be measured.

It is preferable that, in the biological information measuring apparatus of the first aspect of the invention, the first light source and the second light source be configured so as to surround the light receiving section.

It is preferable that, in the biological information measuring apparatus of the first aspect of the invention, the first light source and the second light source be configured in an annular form.

It is preferable that, in the biological information measuring apparatus of the first aspect of the invention, the first light source and the second light source be configured in a circular form.

It is preferable that, in the biological information measuring apparatus of the first aspect of the invention, the first light source and the second light source be configured in a concentrical form.

It is preferable that the biological information measuring apparatus of the first aspect of the invention further include: an uneven filter disposed on an optical path of the transmitting light or an optical path of the reflected light, whose light transmittance is set so that the intensity of the light received by the light receiving elements is uniform in the entire light receiving section.

According to this constitution, the amount of light transmitted through the uneven filter can be uniform over the entire uneven filter. In addition, it is possible to make the light intensity uniform over the light receiving elements constituting the light receiving section regardless of the position from the first light source and the second light source. As a result, the influence in that the light intensity distribution data to be acquired depends on the distance between the light receiving element and the light source can be reduced.

A second aspect of the invention provides a biological information measuring method for measuring information of a living body by irradiating the living body with light, the method including: providing a biological information measuring apparatus including: a first light source irradiating the living body with light; a second light source irradiating the living body with light; and a light receiving section constituted by a plurality of light receiving elements that receives reflected light reflected by the living body or transmitting light transmitting through the living body, during light irradiation of the living body, the first light source and the second light source being disposed so that a first distance between the light receiving section and the first light source is different from a second distance between the light receiving section and the second light source; acquiring first light intensity distribution information by the light receiving section while lighting only the first light source; acquiring second light intensity distribution information by the light receiving section while lighting only the second light source; and acquiring light intensity distribution information of a depth in the living body to a point to be measured by calculating based on the first light intensity distribution information and the second light intensity distribution information.

According to this method, since the two light sources irradiating the light onto the living body and the light receiving section receiving the reflected light that is reflected by the living body or transmitting light transmitting through the living body during light irradiation of the living body is included in the biological information measuring apparatus, and since the first distance between the light receiving section and the first light source is different from the second distance between the light receiving section and the second light source, it is possible to measure a tissue of a living body having depth selectivity.

Furthermore, this constitution is configured so that light intensity distribution information of a depth in the living body to a point to be measured is acquired by calculating in a image processing based on first light intensity distribution information that is acquired when the first light source is only lit and second light intensity distribution information that is acquired when the second light source is only lit. It is thereby possible to simplify the constitution of the biological information measuring apparatus. Also, the biological information measuring apparatus can be decreased in size.

### BRIEF DESCRIPTION OF THE DRAWINGS

FIG. 1 is a block diagram showing the configuration of a first embodiment of the invention;

FIG. 2 is a flow chart showing an operation of the apparatus shown in FIG. 1.

FIG. 3 is a view illustrating the configuration of an uneven filter.

FIG. 4 is a schematic diagram showing the configuration of an object to be measured, which is used in the experiment for verifying an effect.

FIG. 5 illustrates an image of the light intensity distribution acquired as a result of irradiation of light onto a sample shown in FIG. 4.

FIG. 6 illustrates an image of the light intensity distribution acquired as a result of irradiation of light onto a sample shown in FIG. 4.

FIG. 7 is a view showing a result of measurement of a tissue of a living body with depth selectivity in the invention.

FIG. 8 is a block diagram showing the configuration of a second embodiment of the invention.

FIG. 9 is a block diagram showing the configuration of a third embodiment of the invention.

FIG. 10 is a view illustrating the principle of the invention.

FIG. 11 is a view illustrating the principle of the invention.

### DESCRIPTION OF EXEMPLARY EMBODIMENTS

Hereinafter, a biological information measuring apparatus and a biological information measuring method for measuring a tissue of living body with depth selectivity of the invention will be described with reference to the accompanying drawings.

First, a biological information measuring apparatus according to a first embodiment of the invention will be described with reference to the accompanying drawings.

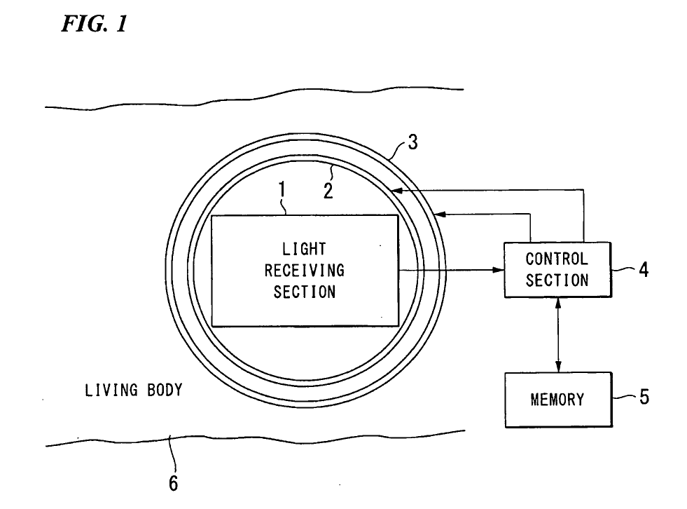

FIG. 1 is a block diagram showing the configuration of the present embodiment.

In FIG. 1, reference numeral 1 denotes a light receiving section configured to include a CCD or the like for acquiring an image.

Reference numerals 2 and 3 denote light sources (hereinafter, referred to as annular light sources) having an annular shape. Each of the light sources 2 and 3 is formed by using a semiconductor laser or an optical fiber bundle. The light source 2 is the first light source of the invention that irradiates light onto the living body. The light source 3 is the second light source of the invention that irradiates light onto the living body.

The light receiving section 1 and the two annular light sources 2 and 3 are arranged so that the middle point (point of intersection of diagonal lines) of the light receiving section 1 and the centers of the two annular light sources 2 and 3 are equal.

Reference numeral 4 denotes a control section that makes an ON/OFF control of the annular light sources 2 and 3, reads received light intensity distribution data (image data, first light intensity distribution data, and second light intensity distribution data) that the light receiving section 1 outputs, and performs image processing.

Reference numeral 5 denotes a memory that stores the received light intensity distribution data (image data, first light intensity distribution data, and second light intensity distribution data).

The light receiving section 1 and the two annular light sources 2 and 3 are provided so as to be in close contact with a living body 6, such as a person's arm.

This makes light of the annular light sources 2 and 3, which is reflected from a surface of the epidermis of the living body 6, not incident into the light receiving section 1.

Furthermore, the light receiving section 1 is constituted by a plurality of light receiving elements. The light receiving elements are the pixels constituting the CCD. In FIG. 1, the light receiving section 1 is configured in a rectangular form. The light receiving section may be configured in a circular form. The light receiving elements receive the reflected light that is reflected by the living body or transmitting light transmitting through the living body, during light irradiation of the living body. The light source 2 and the light source 3 are disposed so that a first distance between the middle point of the light receiving section 1 and the light source 2 is different from a second distance between the middle point of the light receiving section 1 and the light source 3.

As shown in FIG. 1, the annular light sources 2 and 3 are configured so as to surround the light receiving section 1, and are configured in a circular form and configured in a concentrical form. The light receiving section 1 is surrounded by the annular light source 2. Also, since the annular light source 2 has the diameter smaller than the diameter of the annular light source 3, the annular light source 2 is surrounded by the annular light source 3. Therefore, the first distance between the middle point of the light receiving section 1 and the light source 2 is shorter than the second distance between the middle point of the light receiving section 1 and the light source 3.

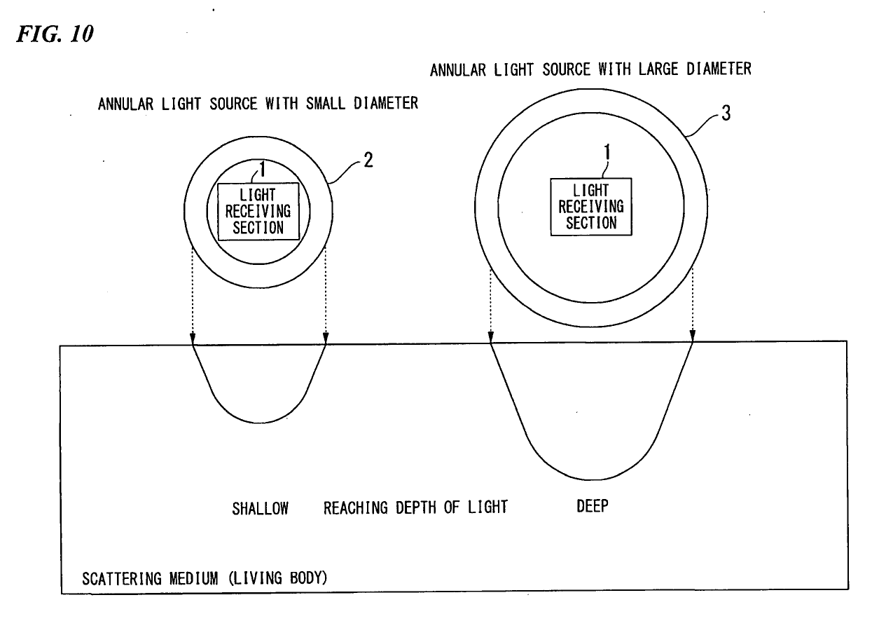

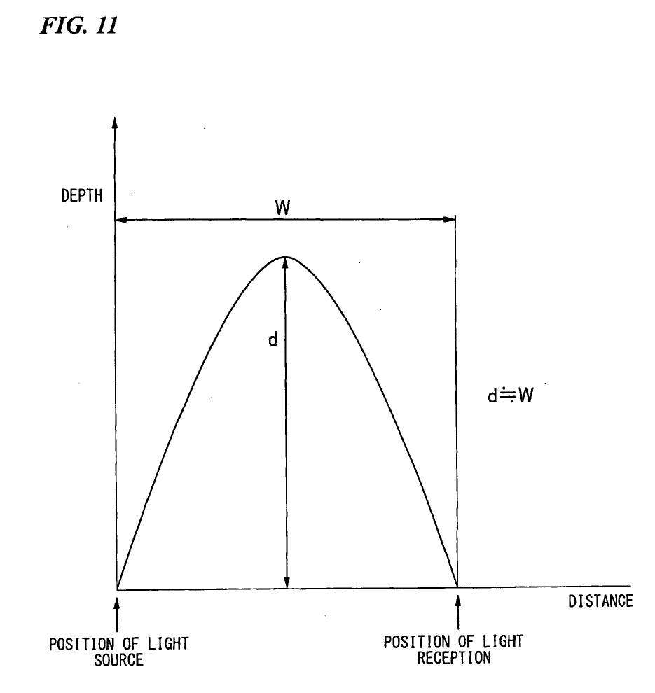

Here, the basic principle of the invention will be described with reference to FIGS. 10 and 11.

FIG. 10 shows a view in which light of the annular light source 2 having a small diameter and light of the annular light source 3 having a great diameter reach up to a predetermined depth in a living body that is a scattering medium and then enters the light receiving section 1.

As shown in FIG. 10, the reaching depth of light emitted from the annular light source having a small diameter is short but the reaching depth of light emitted from the annular light source having a great diameter is deep.

This means that an image positioned at the small depth is obtained when the annular light source 2 having a small diameter is turned on and the image is received by the light receiving section 1. On the other hand, an image positioned at the great depth is obtained in the case when the annular light source 3 having a great diameter is turned on and the image is received by the light receiving section 1.

FIG. 11 shows a view illustrating the relationship among the light receiving position, the light source position, and the reaching depth of light.

As shown in FIG. 11, the reaching depth of light is substantially equal to a distance between the light source position and the light receiving position.

That is, a reaching depth d of light and a distance W between the light source position and the light receiving position have the relationship of d=W.

Accordingly, it is possible to selectively acquire an image, which is positioned at a predetermined depth, by determining the positional relationship between the light receiving section 1 and the annular light sources 2 and 3 on the basis of the relationship.

In the invention, a measurement of a tissue of a living body with depth selectivity is performed by using this principle.

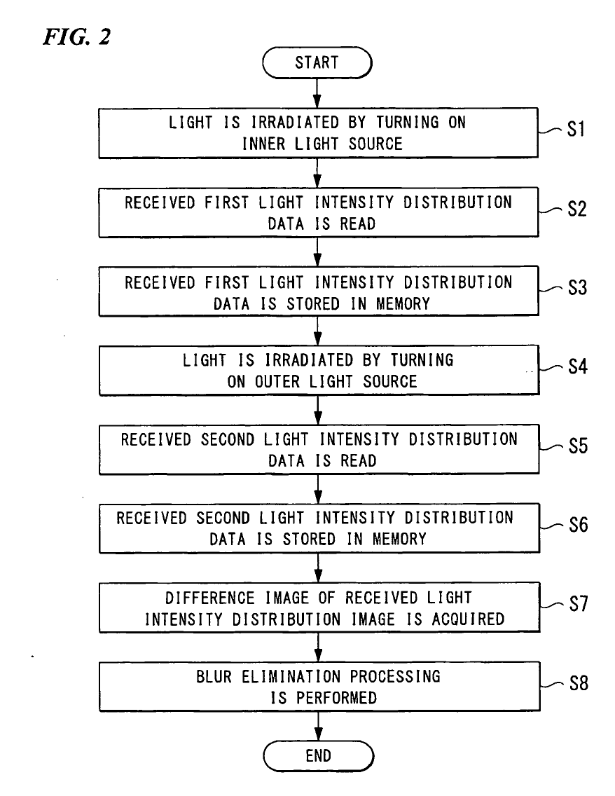

Next, an operation of the biological information measuring apparatus shown in FIG. 1 will be described with reference to FIG. 2.

First, a control section 4 turns on the inner annular light source 2 (light source closer to the light receiving section 1) so as to irradiate light onto the living body 6 (step S1).

At this time, since the annular light source 2 is in close contact with the living body 6, the light emitted from the light source enters the inside of the living body 6 without being reflected from the epidermis.

Then, the control section 4 reads first light intensity distribution data (image data) in which the light receiving section 1 outputs in a state where the annular light source 2 is lit (step S2).

Then, the control section 4 stores the read first light intensity distribution data in a memory 5 (step S3).

Thus, the first light intensity distribution data (image data) is stored in the memory 5 when the inner annular light source 2 is lit.

Then, the control section 4 turns off the annular light source 2 and turns on the outer annular light source 3 (light source distant from the light receiving section 1) so as to irradiate light onto the living body 6 (Step S4).

At this time, since the annular light source 3 is in close contact with the living body 6, light emitted from the light source enters the inside of the living body without being reflected from the epidermis.

Then, the control section 4 reads second intensity distribution data (image data) in which the light receiving section 1 outputs in a state where the annular light source 3 is lit (step S5).

Then, the control section 4 stores the read second light intensity distribution data in the memory 5 (step S6).

Thus, the second light intensity distribution data (image data) is stored in the memory 5 when the outer annular light source 3 is lit. As a result, two kinds of light intensity distribution data are stored in the memory 5.

Then, a difference image is acquired by subtracting the light intensity distribution data, which is obtained by multiplying the first light intensity distribution data when the inner annular light source 2 is lit by a weighting factor, from the second light intensity distribution data when the outer annular light source 3 is lit, which is stored in the memory 5, for every pixel of the light receiving section 1 (step S7).

That is, the difference image is acquired by calculating “(the pixel value of an image when an outer light source is lit)−(the pixel value of an image when an inner light source is lit)×(the weighting factor (for example, 0.8))”.

The control section 4 stores the acquired difference image in the memory 5. This difference image is the image in which a predetermined depth is selected.

Then, with regard to the difference image stored in the memory 5, the control section 4 sets a depth in that a depth-depending point spread function is selected, and performs deconvolution integration processing.

Since this processing is well known blur elimination processing, an explanation thereof will be omitted.

Thus, the depth-selected image in which blur caused by light scattering is eliminated can be acquired.

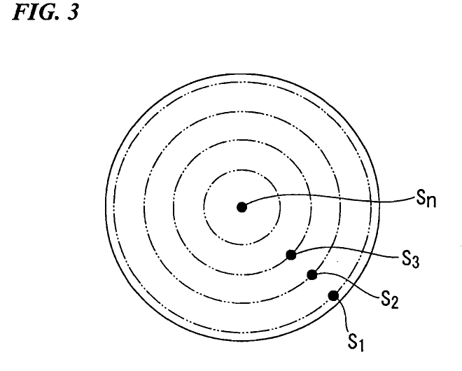

In addition, an uneven filter shown in FIG. 3 may be provided at the light incidence surface of the light receiving section 1 shown in FIG. 1. The light incidence surface is the surface into which the light enters.

The light transmittance of the uneven filter is set so that the intensity of light received by a plurality of light receiving elements provided in the light receiving section 1 is uniform as a whole.

That is, the amount of light transmitted through the uneven filter is set so as to be equal over the entire surface of the uneven filter.

Specifically, as shown in FIG. 3, the light transmittance is set so that the light transmittance is lowest in the uneven filter at the circumferential line S1 of an outermost edge of the uneven filter, and the light transmittance gradually increases from the circumferential line S1 to circumferential lines S2, S3, . . . and Sn in the inward radial direction. In addition, the light transmittance is highest in the uneven filter in a region corresponding to a central point Sn.

Here, the circumferential line S1 of the uneven filter is arranged to be closest to the annular light source 2, and the distance from the annular light source 2 gradually increases with movement toward the circumferential lines S2, S3, . . . and Sn in the inward radial direction. As a result, the distance from the annular light source 2 is greatest in the region corresponding to the central point Sn.

That is, the light transmittance of the uneven filter is set so that the light transmittance is lowest in the uneven filter in a portion disposed at the position closest from the annular light source 2, the light transmittance gradually increases depending on increasing the distance from the annular light source 2, and the light transmittance is highest in the uneven filter in a portion disposed at the position farthest from the annular light source 2 in a state where a biological information measuring apparatus is provided in a living body, such as an arm.

In this constitution, since the amount of light transmitted through the uneven filter can be made uniform over the entire uneven filter, it is possible to make the light intensity uniform over the plurality of light receiving elements provided in the light receiving section 1 regardless of the position from the light sources 2 and 3.

As a result, the influence in that the light intensity distribution data to be acquired depends on the distance between the light receiving element and the light source can be reduced.

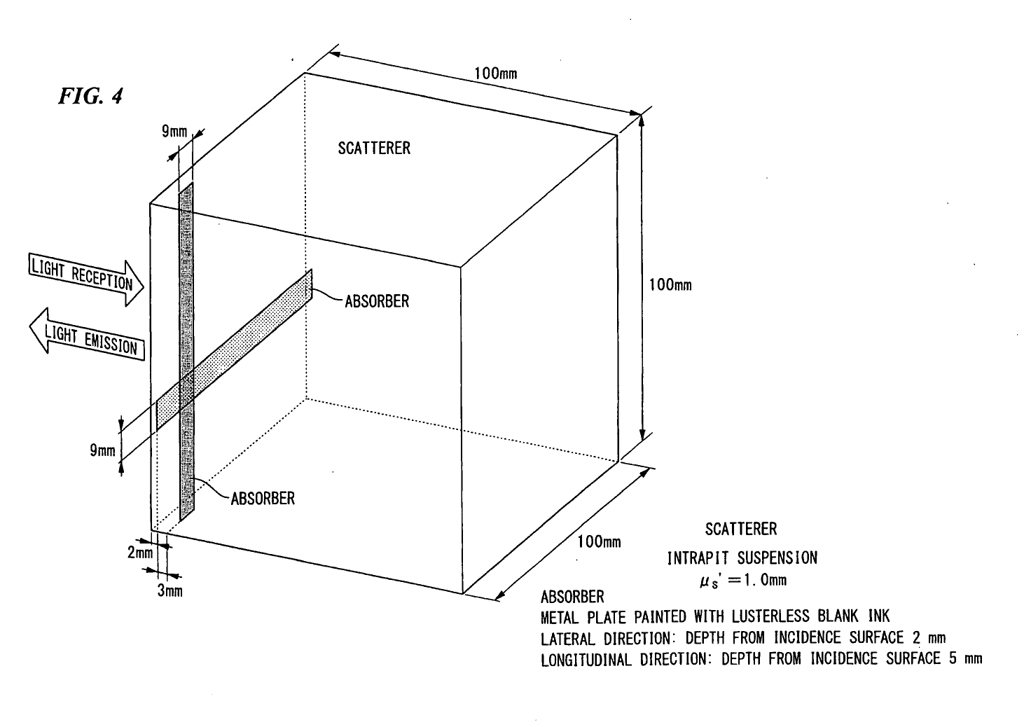

Next, a result of biological information measurement using an experimental apparatus will be described with reference to FIGS. 4 to 7.

FIG. 4 is a schematic diagram showing the configuration of a sample obtained by simulating a living body for verifying the effect of the biological information measuring apparatus shown in FIG. 1.

In FIG. 4, inside a cube having sides of 100 mm, intrepid suspension is injected as a scatterer, and a metal plate painted with lusterless black ink is disposed as an absorber as a blood vessel.

A metal plate extending in the horizontal (lateral) direction is provided at the position corresponding to a depth of 2 mm from a light incidence surface, and a metal plate extending in the vertical (longitudinal) direction is provided at the position corresponding to a depth of 5 mm from the light incidence surface.

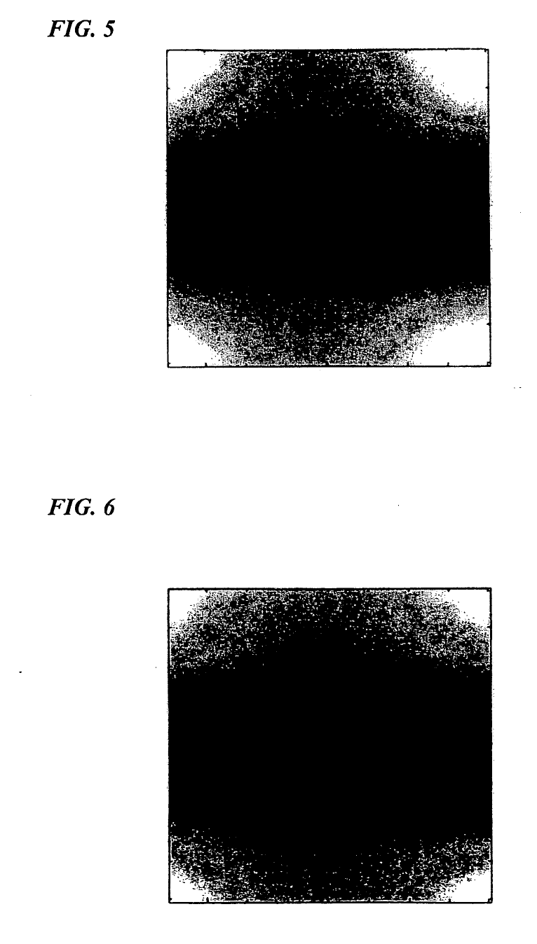

In addition, FIG. 5 shows an image which is the light intensity distribution data of reflected light, and is acquired when an annular light source having a great diameter is turned on.

In addition, FIG. 6 shows an image which is the light intensity distribution data of reflected light, and is acquired when an annular light source having a small diameter is turned on.

Although the difference between FIGS. 5 and 6 is not clear, the absorber (metal plate extending in the longitudinal direction) existing at the deep position (5 mm from the light incidence surface) in the image shown in FIG. 6 is more blurred than the absorber (metal plate extending in the longitudinal direction) shown in FIG. 5.

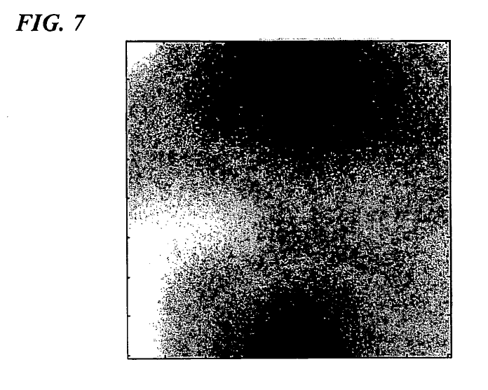

An image obtained by performing the above-described calculation processing on the two images is shown in FIG. 7.

As apparent from FIG. 7, it is observable that an image of the absorber (metal plate extending in the longitudinal direction) positioned at the predetermined depth (5 mm from the light incidence surface in this example) is clear.

In addition, although an example in which two annular light sources are provided has been illustrated in the configuration shown in FIG. 1, three or more annular light sources having different diameters may be provided.

In this manner, since biological information at a position as deep as an increase in the diameter of an annular light source can be acquired, an image of biological information corresponding to a desired depth can be acquired.

As described above, when the light source (annular light source 2) close to the light receiving section 1 is turned on, many light components propagate through a shallow portion in a light-scattering medium, and accordingly, information on the shallow portion is included to a great degree.

On the other hand, in the case of a light source (annular light source 3) far from the light receiving section 1, light propagates through a deep portion in the light-scattering medium, and accordingly, information on the deep portion is included to a great degree.

Accordingly, information corresponding to different depths is included in received light intensity distribution data obtained from the plurality of light sources.

Thus, an absorber of a shallow portion and an absorber of a deep portion can be separately extracted by performing processing for calculating a difference between images of both received light intensity distribution data items, which can be obtained when each of the plurality of light sources is turned on, with a proper weight.

Moreover, it is also possible to increase the number of divisions in the depth direction by increasing the number of light sources.

As described above, the apparatus that measures a tissue of a living body with depth selectivity can be realized with the simple configuration.

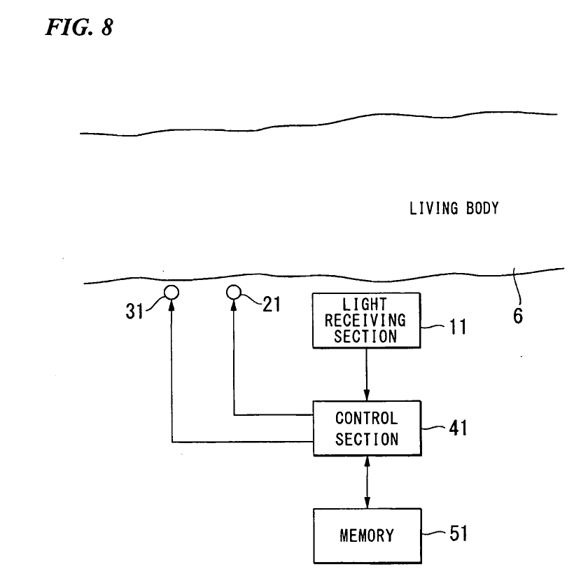

Next, a second embodiment of the invention will be described with reference to FIG. 8.

FIG. 8 is a block diagram showing the configuration of the second embodiment.

The configuration shown in FIG. 8 is different from that shown in FIG. 1 in that two point light sources 21 and 31 (light sources having fine light emitting surfaces; for example, LEDs) are provided instead of two annular light sources.

In this configuration, the characteristics of an uneven filter are also modified for the point light sources.

In this case, the transmittance of the uneven filter is set so that the transmittance is low in a portion of a light incidence surface of a light receiving section 11 close to the two point light sources, and the transmittance gradually increases depending on increasing the distance from the two point light sources.

Moreover, in this configuration, an image at the predetermined depth is obtained by the same operation as that shown in FIG. 2.

Since a detailed operation is the same as that described above, a detailed explanation thereof will be omitted.

According to the configuration shown in FIG. 8, it is possible to use an LED or the like as a light source. As a result, the configuration of the biological information measuring apparatus can be further simplified.

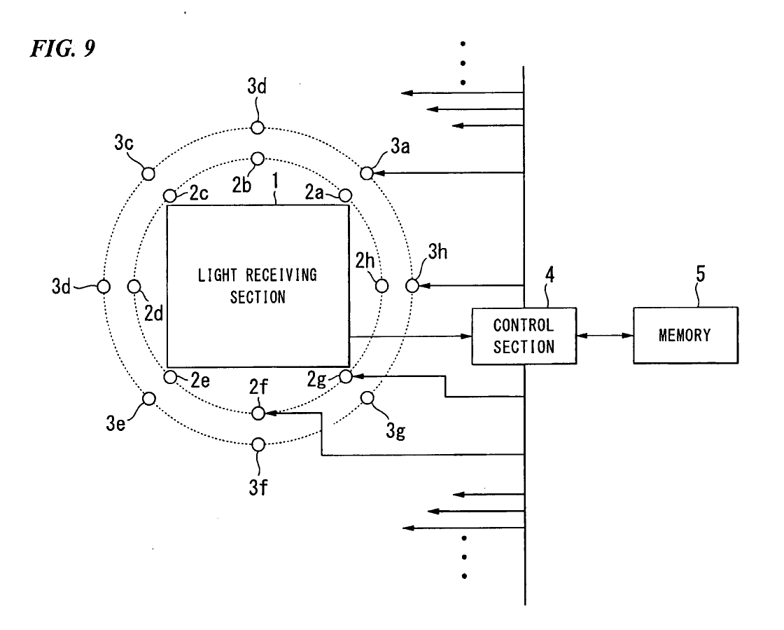

Next, a third embodiment of the invention will be described with reference to FIG. 9.

FIG. 9 is a block diagram showing the configuration of the third embodiment.

The configuration shown in FIG. 9 is different from that shown in FIG. 1 in that an annular light source is realized by using a plurality of (eight) discrete point light sources (light sources having fine light emitting surfaces; for example, LEDs) instead of two annular light sources and the control section 4 conducts an ON/OFF control of each of point light sources 2a to 2h and 3a to 3h.

In the configuration, the characteristics of the uneven filter are also modified for the discrete point light sources.

In this case, the transmittance of the uneven filter is set so that the transmittance is low in a portion of a light incidence surface of the light receiving section 11 close to the eight point light sources, and the transmittance gradually increases depending on increasing the distance from the eight point light sources.

Moreover, in this configuration, an image at the predetermined depth is obtained by the same operation as that shown in FIG. 2.

Since the detailed operation is the same as that described above, a detailed explanation thereof will be omitted.

According to the configuration shown in FIG. 9, it is possible to use an LED or the like as a light source. As a result, the configuration of the biological information measuring apparatus can be further simplified.

In addition, since the point light sources are uniformly disposed around the light receiving section 1, it is possible to make uniform the intensity of light incident into the light receiving section 1.

In addition, the number of point light sources shown in FIG. 9 may also be increased as necessary.

In addition, although double annular light sources are realized by using the discrete point light sources in FIG. 9, annular light sources may be provided greater than or equal to threefold.

In addition, although the annular light source using the discrete point light sources shown in FIG. 9 is realized by eight point light sources, the number of point light sources does not necessarily need to be eight but may be greater than or equal to seven.

In addition, the uneven filter described above may also be realized by image processing in the control section 4.

That is, if emission brightness distribution of a light source and the positional relationship among light receiving elements provided in the light receiving section 1 are known, such information is stored in the memory 5. Then, when received light intensity distribution data (image data) is obtained, a pixel value included in the received light intensity distribution data is corrected by calculation processing based on the stored information on the emission brightness distribution of the light source and the positional relationship among the light receiving elements provided in the light receiving section 1.

Since the uneven filter does not need to be provided in such a manner, it is possible to simplify the configuration of an apparatus.

Moreover, in the case of realizing an uneven filter by image processing, the received light intensity distribution data can be corrected on the basis of the emission brightness distribution of the light source and the positional relationship among the light receiving elements provided in the light receiving section 1. Accordingly, two annular light sources do not need to be concentric.

Since the centers of the two annular light sources become eccentric, it is possible to increase the number of patterns in which the distances between the light receiving element and the light sources are different from each other. An image at an optional depth can thereby be acquired.

As described above, two light sources that irradiate light onto a living body and a light receiving section that receives reflected light which is reflected from the living body, or transmitting light which is transmitted through the living body, when light from the light source is irradiated, are provided. Also, the light sources are disposed so that the first distance between the light receiving section and the first light source is different from the second distance between the light receiving section and the second light source. Accordingly, a measurement of a tissue of a living body can be performed with depth selectivity.

In addition, since light intensity distribution information corresponding to a depth in the living body to a point to be measured is acquired by an operation of image processing based on the first light intensity distribution information acquired when the first light source is only turned on and second light intensity distribution information acquired when the second light source is only turned on, it is thereby possible to simplify the constitution of the biological information measuring apparatus. Also, the biological information measuring apparatus can be decreased in size.

In addition, the biological information measuring apparatuses shown in FIGS. 1, 8, and 9 may be applied to a biometric authentication terminal, a general-purpose small biometric authentication module, a commander such as keyless entry, a remote control, and the like.

In addition, the biological information measuring apparatuses shown in FIGS. 1, 8, and 9 may be provided in electronic apparatuses, such as a computer, a mobile phone, and home appliances.

By making a biometric authentication apparatus provided in an electronic apparatus, restriction of use can be set so that only the owner can use the electronic apparatus, for example.

In addition, the biometric authentication processing may be performed by recording a program for realizing functions of the control sections shown in FIGS. 1, 8, and 9 in a computer-readable recording medium, reading the program recorded in the recording medium into a computer system, and executing the program.

In addition, the “computer system” referred to herein is assumed to include hardware, such as a peripheral device, and so an OS.

In addition, examples of the “computer-readable recording medium” include portable media, such as a flexible disk, a magneto-optic disk, a ROM, or a CD-ROM, and a storage device, such as a hard disk built in a computer system.

Furthermore, an example of the “computer-readable recording medium” also includes a medium that stores a program for a predetermined period of time like volatile memory (RAM) in a computer system, which serves as a server or a client when the program is transmitted through a network, such as the Internet, or a communication line, such as a telephone line.

In addition, the program may be transmitted from a computer system, which has a storage device or the like that stores the program, to other computer systems through a transmission medium or transmission waves in the transmission medium.

Here, the “transmission medium” that transmits a program refers to a medium having a function of transmitting information like a network (communication network), such as the Internet, or a communication line, such as a telephone line.

In addition, the above program may be provided to realize a part of the function described above.

Moreover, the program may be a so-called difference file (difference program) that can realize the above function in combination with a program already recorded in a computer system.

While preferred embodiments of the invention have been described and illustrated above, these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A biological information measuring apparatus for measuring information of a living body by irradiating the living body with light, the apparatus comprising:
a first light source irradiating the living body with light;
a second light source irradiating the living body with light; and
a light receiving section constituted by a plurality of light receiving elements that receives reflected light reflected by the living body or transmitting light transmitting through the living body, during light irradiation of the living body, wherein
the first light source and the second light source are disposed so that a first distance between the light receiving section and the first light source is different from a second distance between the light receiving section and the second light source.

2. The biological information measuring apparatus according to claim 1, wherein
the first distance and the second distance are determined depending on a depth in the living body to a point to be measured.

3. The biological information measuring apparatus according to claim 1, wherein
the first light source and the second light source are configured so as to surround the light receiving section.

4. The biological information measuring apparatus according to claim 3, wherein
the first light source and the second light source are configured in an annular form.

5. The biological information measuring apparatus according to claim 4, wherein
the first light source and the second light source are configured in a circular form.

6. The biological information measuring apparatus according to claim 5, wherein
the first light source and the second light source are configured in a concentrical form.

7. The biological information measuring apparatus according to claim 1, further comprising:
an uneven filter disposed on an optical path of the transmitting light or an optical path of the reflected light, whose light transmittance is set so that the intensity of the light received by the light receiving elements is uniform in the entire light receiving section.

8. A biological information measuring method for measuring information of a living body by irradiating the living body with light, the method comprising:
providing a biological information measuring apparatus including: a first light source irradiating the living body with light; a second light source irradiating the living body with light; and a light receiving section constituted by a plurality of light receiving elements that receives reflected light reflected by the living body or transmitting light transmitting through the living body, during light irradiation of the living body, the first light source and the second light source being disposed so that a first distance between the light receiving section and the first light source is different from a second distance between the light receiving section and the second light source;
acquiring first light intensity distribution information by the light receiving section while lighting only the first light source;
acquiring second light intensity distribution information by the light receiving section while lighting only the second light source; and
acquiring light intensity distribution information of a depth in the living body to a point to be measured by calculating based on the first light intensity distribution information and the second light intensity distribution information.
